(19) United States
(12) Patent Application Publication
(10) Pub. No.: US 2005/0234275 A1
(43) Pub. Date: Oct. 20, 2005

(54) Reduction of naphthalene concentration in aromatic fluids

(76) Inventors: Shifang Luo, Pittsford, NY (US); Edmund John Mozeleski, Califon, NJ (US); Jose` Guadalupe Santiesteban, Baton Rouge, LA (US); Richard Henry Schlosberg, Houston, TX (US); Steven E. Silverberg, Seabrook, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 10/826,057

(22) Filed: Apr. 16, 2004

(51) Int. Cl. C07C 5/10

(52) U.S. Cl. 585/269

(57) ABSTRACT

A process for reducing naphthalene concentration in a naphthalene containing aromatic fluid, the process comprises hydrogenating at least a portion of the naphthalene in the presence of a Group VIII metal catalyst at a temperature from 50° C. to 110° C. to form tetrahydronaphthalene.

### FIELD OF THE INVENTION

The present invention relates to the reduction of naphthalene concentration in aromatic fluids by hydrogenation. More particularly, the present invention relates to the selective hydrogenation of naphthalene to tetrahydronaphthalene.

### BACKGROUND

Large-scale refinery production separates crude oil into many fractions one of which is known as virgin naphtha. Virgin naphtha is often reformed to make aromatic naphtha or reformate for motor gasoline blending and chemicals recovery. The fractionation process is often a complex distillation that primarily relies on the difference in the boiling points of the components of the reformate for separation into various fractions. Many of these fractions may contain naphthalene. Consequently, there exists a need for aromatic fluids having a reduced naphthalene concentration and a process for producing aromatic fluids having a reduced concentration of naphthalene. These processes permit naphthalene-containing aromatic fluid to be converted into products with enhanced value and to recover by-products for recycling. The reduction of naphthalene in aromatic fluids results in better low temperature performance, reduction of odor, and reduction of volatility.

Japanese Patent Application No. 49-49947 discloses a method of selective hydrogenation of naphthalene to tetrahydronaphthalene by hydrogenating in the presence of one or more supported oxides or sulfides of metals of Group VIII or Group VIB and hydrogen containing hydrogen sulfide or a gas containing hydrogen.

“Selective Hydrogenation of Naphthalene and Alkylnaphthalenes on a Palladium Catalyst”, Inst. Geol. Razrab., Moscow, U.S.S.R., (1972), discloses a method of selectively hydrogenating naphthalene to tetrahydronaphthalene in the presence of a 0.5% palladium on alumina catalyst in sulfidated form at a temperature from 400° C. to 500° C. and a pressure of 25 atm. to 100 atm. The article discloses that at lower temperature or higher pressures decalins or decahydronaphthalene are favored.

“Selective Hydrogenation of Naphthalene to Tetralin”, Inst. Goryuch. Iskop, Moscow, U.S.S.R., (1982), discloses a method of selectively hydrogenating naphthalene to tetrahydronaphthalene in the presence of an aluminum-palladium sulfide catalyst in the presence of a maximum of 0.15% sulfur at a pressure of 5.0 MPa and a temperature of 260° C.

“Hydrogenation of Naphthalene to Tetralin in the Presence of a Palladium Catalyst”, Inst. Goryuch. Iskop, Moscow, U.S.S.R., (1972), discloses a method of selectively hydrogenating naphthalene to tetrahydronaphthalene in the presence of a palladium on aluminum oxide in sulfated form in the presence of no more than 0.25% sulfur compounds at a temperature of 200° C. to 280° C.

“Selectivity in Platinum Metal Catalyzed Hydrogenations”, Engelhard Industries, Technical Bulletin, (1965), discloses that a palladium catalyst in a hydrogenation of naphthalene reaction at 1000 psig and 115° C. to 120° C. spontaneously stops at the formation of tetrahydronaphthalene.

It would be desirable to develop new processes that maintain or increase conversion of naphthalene in hydrocarbon fluid streams to tetrahydronaphthalene while maintaining or improving process economics through lower operating pressure and/or temperature.

### SUMMARY OF THE INVENTION

The present invention provides processes for reducing the naphthalene concentration in a naphthalene containing aromatic fluid. The aromatic fluid comprises at least one aromatic compound, typically a mixture of two or more aromatic compounds.

One embodiment of the present invention provides a process for reducing naphthalene concentration in a naphthalene containing aromatic fluid, the process comprises hydrogenating at least a portion of the naphthalene in the presence of a Group VIII metal catalyst at a temperature from 50° C. to 110° C. to form tetrahydronaphthalene.

One embodiment of the present invention provides a process for reducing naphthalene concentration in a naphthalene containing aromatic fluid, the process comprising hydrogenating at least a portion of the naphthalene in the presence of a Group VIII metal catalyst, preferably a supported Group VIII metal catalyst, at a temperature from 50° C. to 110° C., alternatively from 90° C. to 105° C., to form tetrahydronaphthalene, and preferably further hydrogenating the tetrahydronaphthalene to decahydronaphthalene. In a preferred embodiment of this embodiment, the Group VIII metal is palladium, and the catalyst is most preferably a supported palladium catalyst, preferably where the support is selected from alumina, carbon, and silica. In another preferred embodiment, where a supported palladium catalyst is utilized, 0.01 wt % to 25 wt %, preferably 0.1 wt % to 1.0 wt % of the palladium catalyst on an alumina support or silica support is utilized. In an embodiment where the support is a carbon support and the Group VIII metal is palladium, it is preferable to utilize from 0.01 wt % to 25 wt %, preferably 0.1 wt % to 1.2 wt %, palladium catalyst on the carbon support. The weight % is based on the total weight of the catalyst.

In any of the above embodiments, the hydrogenation occurs in either a fixed bed reactor or a batch reactor at a pressure from 100 psig (690 Kpag) to 3500 psig (2413 Kpag), alternatively from 250 psig (1724 Kpag) to 500 psig (3448 Kpag). In yet another embodiment, of any of the above embodiments, the naphthalene containing aromatic fluid comprises at least 0.2 wt % naphthalene, preferably in the ranges of from 0.5 wt % to 35 wt % naphthalene, preferably from 1 wt % to 30 wt % naphthalene, more preferably from 5 wt % to 15 wt % naphthalene, and most preferably from 8 wt % to 12 wt % naphthalene. The weight % is based on the total weight of the aromatic fluid.

In one preferred embodiment conversion of naphthalene to tetrahydronaphthalene is greater than 85%, preferably greater than 95%, and more preferably greater than 99%. In an embodiment, the selectivity to tetrahydronaphthalene is greater than 80%, preferably greater than 85%, more preferably greater than 95%, and most preferably greater than from 98%.

### DETAILED DESCRIPTION OF THE INVENTION

The present invention is directed to processes for reducing naphthalene concentration in an aromatic fluid.

One embodiment of the present invention provides a process for reducing naphthalene concentration in a naphthalene containing aromatic fluid, the process comprises hydrogenating at least a portion of the naphthalene in the presence of a Group VIII metal catalyst at a temperature from 50° C. to 110° C. to form tetrahydronaphthalene.

In one embodiment according to the present invention the naphthalene-containing aromatic fluid is exemplified by, but not limited to, Aromatic 150 and Aromatic 200 Fluids sold by ExxonMobil Chemical Company.

The elemental groups herein referred to are based on the CAS version of the Periodic Table of the Elements.

Feedstock

In one embodiment the aromatic fluid containing naphthalene useful in this process can be derived from a substantially dealkylated feedstock. The type of aromatic fluid feedstock useful in one embodiment of the present invention comprises one or more fused-ring polycyclic aromatic compounds, although assemblies of two or more cyclic compounds, either single ring cyclics or aromatics or fused compounds may also be present. In one embodiment according to the present invention the aromatic fluid comprises 1,2,4-trimethylbenzene; 1,2,3-trimethylbenzene; m-cymene; a mixture of alkylbenzene compounds having from 1 to 4 alkyl substituents, each alkyl substituent having from 1 to 4 carbon atoms and the alkylbenzene compounds have a total number of carbon atoms ranging from 10 to 12; naphthalene; and methylnaphthalene.

The polycyclic aromatic compound is typically obtained from catalytic reforming operations but may also be obtained from cracking operations, e.g. fluidized bed catalytic cracking (FCC) or moving bed Thermofor catalytic cracking (TCC). Typically, these feed stocks have a hydrogen content of no greater than about 12.5 wt. % and API gravity no greater than 25 and an aromatic content no less than 50 wt. %.

A substantially dealkylated feedstock is a product that was formerly an alkyl aromatic compound, or mixture of alkyl aromatic compounds, that contained bulky relatively large alkyl group side chains affixed to the aromatic moiety. The dealkylated product is the aromatic compound having substantially no bulky side chain alkyl group. Representative examples of the aromatic compound include phenanthrene, anthracene, dibenzothiophene, fluoroanthene, fluorene, benzothiophene, acenaphthene, biphenyl or naphthalene.

During acid catalyzed cracking and similar reactions, prior dealkylation generally will remove side chains of greater than 5 carbon atoms while leaving behind primarily methyl or ethyl groups on the aromatic compounds. Thus, for purposes of this invention, the polycyclic aromatic compounds can include substantially dealkylated aromatic compounds which contain small alkyl groups, such as methyl and sometimes ethyl and the like, remaining as side chains, but with relatively few large alkyl groups, e.g. the C₃ to C₉ groups.

In one embodiment, the aromatic fluid feedstock comprises a mixture of polycyclic compounds, dealkylated or substantially dealkylated, which would be found in a refinery by-product stream. Alternatively, the aromatic fluid feedstock comprises a relatively pure feed consisting essentially of one type of polycyclic aromatic compound.

Representative examples of suitable polycyclic aromatic refinery by-product derived feedstocks include reformate, light cycle oils and heavy cycle oils from catalytic cracking or pyrolysis processes. Other examples of suitable feedstocks include the liquid product from a delayed or fluid bed coking process, such as a coker gas oil, an aromatics-rich fraction produced by lubricant refining, e.g., furfural extraction. Other sources of suitable feedstocks include a heavy crude fraction obtained by crude fractional distillation.

The polycyclic aromatic compound contemplated contains, but is not limited to, at least 2 cyclic groups and up to at least 5 cyclic groups. It can be a hydrocarbon containing up to 5 or more benzene rings in any arrangement including fixed benzene rings in linear arrangement. It can be almost entirely or predominantly carbocyclic and can include or be part of a heterocyclic system in which at least one of the cyclic elements of the molecule contains at least one heteroatom such as sulfur, nitrogen and/or oxygen.

In one embodiment according to the present invention the mixture of aromatic compounds may be Aromatic 150 or Aromatic 200 fluids sold by ExxonMobil Chemical Company. Aromatic 150 fluid comprises approximately fifty components with some of the principle components comprising about 1.7 wt. % of 1,2,4-trimethylbenzene; about 3.0 wt. % of 1,2,3-trimethylbenzene and meta-cumene; a mixture of about 81.6 wt. % C₁₀ to C₁₂ benzene compounds, having one or more substituents selected from methyl, ethyl, propyl, and butyl; about 8.6 wt. % naphthalene; and about 0.3 wt. % methylnaphthalene. The weight % is based on the total weight of the fluid.

Alternatively, the Aromatic 150 fluid may be distilled at atmospheric pressure to remove about 60 wt % of the lighter components to leave an Aromatic 150 fluid concentrate that is about 40 wt % of the total material prior to distillation. The Aromatic 150 fluid concentrate comprises about 20.4 wt % naphthalene.

Aromatic 200 fluid comprises approximately 25 to 30 components with some of the principle components comprising naphthalene (10 wt %); various alkylnaphthalenes (75 wt %), including 2-methylnaphthalene (26 wt %), 1-methylnaphthalene (13 wt %), 2-ethylnaphthalene (2 wt %), dimethyl naphthalenes (18 wt %), and trimethyl naphthalenes (7 wt %); and the remaining 15 wt % comprises primarily alkylbenzenes, as determined by gas chromatographic analysis. The weight % is based on the total weight of the fluid.

In one embodiment of the present invention the aromatic fluid includes, but is not limited to, Aromatic 150; Aromatic 200; aromatic refinery by-product derived feedstocks including, but not limited to, reformate, light cycle oils and heavy cycle oils from catalytic cracking or pyrolysis processes; the liquid product from a delayed or fluid bed coking process, such as a coker gas oil; an aromatics-rich fraction produced by lubricant refining, e.g., furfural extraction; heavy crude fraction obtained by crude fractional distillation; coal tar; and asphaltenes. In another embodiment of the present invention the aromatic fluid includes any feedstock which comprises naphthalene and alkyl-benzenes; alternatively the aromatic fluid comprises at least 0.2 wt. % naphthalene; alternatively the aromatic fluid comprises from about 0.2 wt % to about 50 wt % naphthalene; alternatively the aromatic fluid comprises from about 0.5 wt % to about 35 wt % naphthalene; alternatively the aromatic fluid comprises from about 1 wt % to about 30 wt % naphthalene; alternatively the aromatic fluid comprises from about 5 wt % to about 15 wt % naphthalene; alternatively the aromatic fluid comprises from about 8 wt % to about 12 wt % naphthalene. The weight % is based on the total weight of the aromatic fluid.

Hydrogenation

One embodiment of the present invention provides for a process of reducing the concentration of naphthalene in an aromatic fluid by hydrogenation.

One embodiment of the present invention provides a process for reducing naphthalene concentration in a naphthalene containing aromatic fluid, the process comprises hydrogenating at least a portion of the naphthalene in the presence of a Group VIII metal catalyst at a temperature from 50° C. to 110° C. to form tetrahydronaphthalene. Group VIII metal catalysts includes the active metals, the metal oxides, and the metal sulfides. The term “active metal” means the zero valent form of the metal, i.e., the elemental metal. Preferably, the Group VIII metal catalyst is the active metals. More preferably, the Group VIII metal catalyst is palladium.

In one embodiment the hydrogenation may occur over a supported catalyst. The support used could be those known in the art such as alumina, silica, carbon, silica-alumina, clay, zirconia, titania, microporous materials, with pore mouths large enough to allow reactant accessibility to the metal sites, such as molecular sieves, including crystalline zeolites (aluminosilicates), aluminophosphates (AIPOs), metalloaluminophosphates (MeAPOs), and silicoaluminophosphates (SAPOs), and mesoporous materials of the MCM-41 family. Preferred supports are those such as alumina, carbon or silica, including, but not limited to, supports whose Bronsted acidity has been reduced by an inorganic or organic base compound. The catalyst may be chosen from, but not limited to, about a 0.01 wt % to 25 wt % palladium catalyst on an alumina support, alternatively from about a 0.1 wt % to 5 wt % palladium catalyst on an alumina support, alternatively from about a 0.1 wt % to 1.2 wt % palladium catalyst on an alumina support, alternatively from about a 0.5 wt % palladium catalyst on an alumina support; alternatively about a 0.01 wt % to 25 wt % palladium catalyst on a carbon support, alternatively from about a 0.1 wt % to 5 wt % palladium catalyst on a carbon support, alternatively from about a 0.1 wt % to 1.2 wt % palladium catalyst on a carbon support, alternatively from about a 1.0 wt % palladium catalyst on a carbon support; and alternatively about a 0.01 wt % to 25 wt % palladium catalyst on a silica support, alternatively from about a 0.1 wt % to 5 wt % palladium catalyst on a silica support, alternatively from about a 0.1 wt % to 1.2 wt % palladium catalyst on a silica support, alternatively from about a 0.5 wt % palladium catalyst on a silica support. The weight % is based on the total weight of the catalyst.

With most catalysts, the following reaction conditions can be used in any of the embodiments disclosed herein. Temperatures may typically range from about 50° C. to 250° C., alternatively from about 75° C. to 200° C., alternatively from about 85° C. to 150° C., alternatively from about 90° C. to 125° C., alternatively at about 100° C. Pressures may typically range from about 100 psig to 3500 psig, alternatively from about 125 psig to 2000 psig, alternatively from about 150 psig to 1000 psig, alternatively from about 200 psig to 500 psig, alternatively from about 250 psig to 400 psig, alternatively at about 300 psig. The reaction may take place in either a fixed-bed reactor or a batch reactor.

Reduction of Naphthalene

In another embodiment, the naphthalene conversion via hydrogenation is greater than from 85%, alternatively greater than from 90%, alternatively greater than from 95%, alternatively greater than from 99%, alternatively greater than from 99.5%. In another embodiment, the selectivity to tetrahydronaphthalene via hydrogenation is greater than from 80%, alternatively greater than from 85%, alternatively greater than from 95%, alternatively greater than from 98%, alternatively greater than from 99%.

### EXAMPLES

Test Methods

Samples of reaction mixtures were withdrawn at regular time intervals and analyzed by gas chromatography (GC) (non-polar HP-1 column, 30 m, crosslinked methylsiloxane) and GC-Mass Spectrometry (GC/MS), using the same column. A Hewlett Packard HP 6890 Series GC System and GC/MS System were utilized.

Fluorescent Indicator Adsorption (FIA) was measured by ASTM method D-1319.

The weight % of the metal of the catalyst is based on the total weight of the catalyst. The weight % of the components of the aromatic fluid are based on the total weight of the aromatic fluid.

### Examples 1, 2, & 3

### Hydrogenation in a Batch Reactor

A 300 cc Eze-seal autoclave from Autoclave Engineers equipped with a Robinson-Mahoney catalyst basket was utilized for the batch experiments. The catalyst basket was first charged with the palladium catalyst and the basket was then inserted into the body of the autoclave. The autoclave was sealed and charged with a mixture of 1,2,4-trimethylbenzene and naphthalene concentrate. The naphthalene concentrate was generated by distillation of the lighter boiling components of Aromatic 150 Fluid and contained naphthalene (55.4 wt. %) and polyalkylbenzenes (balance). The resulting feed composition in the reactor contained 1,2,4-trimethylbenzene (48.8 wt %), polyalkylbenzenes from Aromatic 150 Fluid (24.3 wt. %) and naphthalene (26.9 wt. %). The contents of the autoclave were purged with hydrogen then pressurized to approximately 250 psig with hydrogen. The stirred mixture was then heated to 100° C., at 100° C., the pressure was adjusted to approximately 300 psig H₂ and maintained at approximately 300 psig H₂ for 4 hours. The feed to catalyst ratio was 10 to 1 (g/g). GC and GC/MS samples were removed to monitor the reaction.

Two different catalysts were tested: a commercial 0.5 wt. % palladium catalyst on alumina, 3.2 mm pellets obtained from Aldrich Chemical Co. Catalog No. 20,574-5, and a commercial 1 wt. % palladium catalyst on carbon, 4 to 8 mesh obtained from Aldrich Chemical Co. Catalog No. 20,575-3. The results are shown in Table 1 for the palladium on alumina catalyst and in Table 2 for the palladium on carbon catalyst.

TABLE 1
Pd/Al₂O₃ Catalyst: Composition of
reaction products vs. reaction time

| | Reaction Time (h) | | | | | |
|---|---|---|---|---|---|---|
| Composition (wt %) | Feed | 0.5 h | 1 h | 2 h | 3 h | 4 h |
| 1,2,4-trimethylbenzene | 48.7 | 48.2 | 48.2 | 48.0 | 48.2 | 48.2 |
| A-150 Polyalkylbenzenes | 24.8 | 24.4 | 24.2 | 26.1 | 26.2 | 26.2 |
| Naphthalene | 26.5 | 23.4 | 19.7 | 8.6 | 1.4 | 0.09 |
| Tetrahydronaphthalene | 0.0 | 4.0 | 7.9 | 17.2 | 24.1 | 25.1 |
| Decahydronaphthalene | 0.00 | 0.00 | 0.01 | 0.01 | 0.04 | 0.33 |
| (cis & trans) | | | | | | |
| Conversion (%) | | | | | | 99.70 |
| Selectivity (%) | | | | | | 98.70 |

TABLE 2
Pd/C Catalyst: Composition of reaction product vs. reaction time

| | Reaction Time (h) | | | | |
|---|---|---|---|---|---|
| Composition (wt %) | Feed | 0.25 h | 0.5 h | 1 h | 2 h |
| 1,2,4-trimethylbenzene | 48.6 | 48.7 | 48.9 | 48.8 | 48.7 |
| Polyalkylbenzenes from A150 | 24.5 | 24.1 | 25.9 | 25.7 | 25.8 |
| Naphthalene | 26.9 | 16.1 | 7.7 | 0.1 | 0.1 |
| Tetrahydronaphthalene | 0.0 | 11.2 | 17.5 | 25.4 | 24.9 |
| Decahydronaphthalene | 0.00 | 0.01 | 0.01 | 0.06 | 0.67 |
| (cis & trans) | | | | | |
| Conversion (%) | | | | 99.60 | |
| Selectivity (%) | | | | 99.80 | |

The above procedure was followed again, except the reactants were a combination of Aromatic 200 Fluid containing naphthalene (10.3 wt. %), 2-methylnaphthalene (26.3 wt. %), and 1-methylnaphthalene (12.9 wt. %). No 1,2,4-trimethylbenzene was added. The reactants were reacted with the 0.5 wt. % palladium catalyst on alumina as described above. The results are shown in Table 3 below.

TABLE 3
Pd/Al₂O₃ Catalyst: Composition of
reaction products vs. reaction time

| | Reaction Time (h) | | | | |
|---|---|---|---|---|---|
| Composition (wt %) | Feed | 1 h | 3 h | 5 h | 8 h |
| Naphthalene | 10.3 | 8.1 | 4.6 | 2.1 | 0.34 |
| 1-Methylnaphthalene | 12.9 | 12.4 | 11.4 | 10 | 7.5 |
| 2-Methylnaphthalene | 26.2 | 25.5 | 23.6 | 21.4 | 17.4 |
| Tetrahydronaphthalene | 0.0 | 2.3 | 5.5 | 8.1 | 9.8 |
| Conversion (%) | | | | | 96.70 |

### Example 4

### Hydrogenation in a Fixed-Bed Reactor

A commercial 0.3 wt. % palladium on alumina catalyst was placed in an isothermal fixed-bed reactor of a continuous flow hydrotreater. Aromatic 150 Fluid containing 10.1 wt. % naphthalene and >99 vol % total aromatic was processed at 99-107° C., 300 psig, and a hydrogen to naphthalene molar ratio of 4. The liquid hourly space velocity (LHSV) varied from 1.0 to 6.0 hr⁻¹. The results are shown in Table 4 below.

TABLE 4
Pd/Al₂O₃ Catalyst: Composition of reaction products vs. LHSV

| | LHSV (1/hr) | | | | |
|---|---|---|---|---|---|
| | Feed | 1 | 2 | 4 | 6 |
| Naphthalene, wt % | 10.1 | 0.01 | <0.01 | <0.01 | 0.02 |
| Tetrahyrdonaphthalene, wt % | 0.6 | 10.5 | 10.7 | 10.5 | 11.3 |
| Conversion (%) | | | | | 99.8 |
| Aromatics, FIA vol % | >99 | >99 | | >99 | |

### Examples 5 & 6

### Hydrogenation Comparatives

A hydrogenation reaction was performed over a NiMo hydrotreating catalyst. A feed consisting of naphthalene (33 wt %), tetrahydronaphthalene, and decahydronaphthalene was hydrogenated in two fixed-bed reactors in series at 200-275° C., 50-60 atm hydrogen pressure, 1.0 LHSV, and 50% excess hydrogen. Results are tabulated in Table 5.

TABLE 5
Hydrogenation of naphthalene over a NiMo Catalyst

| | |
|---|---|
| LHSV (1/h) | 1.0 |
| Composition (wt %) | 0.01 |
| Naphthalene Conversion (%) | 85 |
| Selectivity (%) | |
| Tetrahyrdronaphthalene | 90 |
| Decahydronaphthalene | 10 |

A hydrogenation reaction was performed over a Ni metal catalyst. Naphthalene, diluted in n-paraffins to 35 wt %, was hydrogenated over a Ni metal catalyst in a fixed-bed pilot hydrotreater at 220 psig and a hydrogen to naphthalene molar ratio of 4. Results are tabulated in Table 6.

TABLE 6
Naphthalene conversion and selectivity vs.
temperature over a Ni Metal Catalyst

| | Temperature (° C.) | | |
|---|---|---|---|
| | 80 | 105 | 105 |
| LHSV | 3 | 1 | 6 |
| Naphthalene Conversion (%) | 100 | 99.9 | 100 |
| Selectivity (%) | | | |
| tetrahydronaphthalene | 72.7 | 0.4 | 84.7 |
| decahydronaphthalene | 27.3 | 99.6 | 15.3 |

The present invention may be embodied in other specific forms without departing from its spirit or essential characteristics. The described embodiments are to be considered in all respects as illustrative only and not restrictive. The scope of the invention is, therefore, indicated by the appended claims rather than by the foregoing description. All changes which come within the meaning and range of equivalency of the claims are to be embraced within their scope.

1. A process for reducing naphthalene concentration in a naphthalene containing aromatic fluid, the process comprising hydrogenating at least a portion of the naphthalene in the presence of a Group VIII metal catalyst at a temperature from 50° C. to 110° C. to form tetrahydronaphthalene.

2. The process of claim 1 wherein the tetrahydronaphthalene is further hydrogenated to decahydronaphthalene.

3. The process of claim 1 wherein the metal catalyst comprises palladium.

4. The process of claim 1 wherein the metal catalyst is supported.

5. The process of claim 4 wherein the support is selected from alumina, carbon, silica, and mixtures thereof.

6. The process of claim 5 wherein the metal catalyst comprises palladium on an alumina support.

7. The process of claim 6 wherein the metal catalyst comprises 0.01 wt % to 25 wt % palladium on an alumina support.

8. The process of claim 7 wherein the metal catalyst comprises 0.1 wt % to 1.0 wt % palladium on an alumina support

9. The process of claim 5 wherein the metal catalyst comprises palladium on a carbon support.

10. The process of claim 9 wherein the metal catalyst comprises 0.01 wt % to 25 wt % palladium on a carbon support.

11. The process of claim 10 wherein the metal catalyst comprises 0.1 wt % to 1.2 wt % palladium on a carbon support.

12. The process of claim 5 wherein the metal catalyst comprises palladium on a silica support.

13. The process of claim 12 wherein the metal catalyst comprises 0.01 wt % to 25 wt % palladium on a silica support

14. The process of claim 13 wherein the metal catalyst comprises 0.1 wt % to 1.0 wt % palladium on a silica support.

15. The process of claim 1 wherein the hydrogenation occurs at a temperature from 90° C. to 105° C.

16. The process of claim 1 wherein the hydrogenation occurs at a pressure from 100 psig to 3500 psig.

17. The process of claim 16 wherein the hydrogenation occurs at a pressure from 250 psig to 500 psig.

18. The process of claim 1 wherein the hydrogenation occurs in a reactor selected from a fixed bed reactor and a batch reactor.

19. The process of claim 1 wherein the naphthalene contain aromatic fluid comprises from at least 0.2 wt % naphthalene.

20. The process of claim 19 wherein the naphthalene containing aromatic fluid comprises from 0.5 wt % to 35 wt % naphthalene.

21. The process of claim 19 wherein the naphthalene containing aromatic fluid comprises from 1 wt % to 30 wt % naphthalene,

22. The process of claim 19 wherein the naphthalene containing aromatic fluid comprises from 5 wt % to 15 wt % naphthalene.

23. The process of claim 19 wherein the naphthalene containing aromatic fluid comprises from 8 wt % to 12 wt % naphthalene.

24. The process of claim 1 wherein naphthalene conversion to tetrahydronaphthalene is greater than from 85%.

25. The process of claim 24 wherein naphthalene conversion to tetrahydronaphthalene is greater than from 95%.

26. The process of claim 25 wherein naphthalene conversion to tetrahydronaphthalene is greater than from 99%.

27. The process of claim 1 wherein selectivity to tetrahydronaphthalene is greater than from 80%.

28. The process of claim 27 wherein selectivity to tetrahydronaphthalene is greater than from 85%.

29. The process of claim 28 wherein selectivity to tetrahydronaphthalene is greater than from 95%.

30. The process of claim 29 wherein selectivity to tetrahydronaphthalene is greater than from 98%.
